(19) United States
(12) Patent Application Publication
(10) Pub. No.: US 2013/0178670 A1
(43) Pub. Date: Jul. 11, 2013

(54) HYDROGENATION CATALYSTS WITH BULK MULTIPLE OXIDATED SUPPORTS

(71) Applicant: Celanese International Corporation, Irving, TX (US)

(72) Inventors: Zhenhua Zhou, Houston, TX (US); Heiko Weiner, Pasadena, TX (US)

(73) Assignee: Celanese International Corporation, Irving, TX (US)

(21) Appl. No.: 13/734,570

(22) Filed: Jan. 4, 2013

Related U.S. Application Data
(63) Continuation-in-part of application No. 13/595,365, filed on Aug. 27, 2012.
(60) Provisional application No. 61/583,874, filed on Jan. 6, 2012.

(51) Int. Cl. B01J 32/00; C07C 29/149

(52) U.S. Cl. CPC B01J 32/00; C07C 29/149
USPC 568/885; 502/305; 502/310; 502/313; 502/242; 502/254; 502/318; 502/316; 502/312; 502/315; 502/309; 502/307; 502/319; 502/321; 502/303; 502/304

(57) ABSTRACT

The present invention relates to catalysts, to processes for making catalysts and to chemical processes employing such catalysts. The catalysts are preferably used for converting acetic acid and ethyl acetate to ethanol. The catalyst comprises an extruded modified support, and a precious metal. The processes for making the catalysts comprises modifying the catalyst, extruding the catalyst, and impregnating the precious metal onto the catalyst.

### CROSS REFERENCE TO RELATED APPLICATION

This application is a continuation-in-part of U.S. application Ser. No. 13/595,365, filed Aug. 27, 2012, which claims priority to U.S. Provisional App. No. 61/583,874, filed on Jan. 6, 2012, the entireties of which are incorporated herein by reference.

### FIELD OF THE INVENTION

The present invention relates to processes for forming catalysts used in hydrogenation reactions. The present invention also relates to processes for producing ethanol from a feed stream comprising acetic acid and/or an ester thereof in the presence of a catalyst prepared by the inventive catalyst formation methods.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulosic materials, such as corn or sugar cane. Conventional methods for producing ethanol from petrochemical feed stocks, as well as from cellulosic materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulosic material, are converted to ethanol by fermentation. However, fermentation is typically used for consumer production of ethanol, which is suitable for fuels or human consumption. In addition, fermentation of starchy or cellulosic materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. The reduction of various carboxylic acids over metal oxides has been proposed by EP0175558 and U.S. Pat. No. 4,398,039. A summary some of the developmental efforts for hydrogenation catalysts for conversion of various carboxylic acids is provided in Yokoyama, et al., “Carboxylic acids and derivatives” in: Fine Chemicals Through Heterogeneous Catalysis, 2001, 370-379.

U.S. Pat. No. 6,495,730 describes a process for hydrogenating carboxylic acid using a catalyst prepared by impregnating a carbonaceous material with a concentrated aqueous zinc chloride solution, followed by calcinations. The activated carbon, prior to impregnation, may have specific pore volume and surface area. U.S. Pat. No. 6,204,417 describes another process for preparing aliphatic alcohols by hydrogenating aliphatic carboxylic acids or anhydrides or esters thereof or lactones in the presence of a catalyst comprising Pt and Re. The catalyst is obtainable by reducing an aqueous suspension and/or solution of oxides, oxide hydrates, carbonates, nitrates, carboxylates, chelates, sulfates, phosphates and/or halides of Pt, Re and at least one further element from group 5 to 12 and 14 and the lanthanides of the Periodic Table of the Elements. The catalyst may be supported or unsupported.

U.S. Pat. No. 5,149,680 describes a process for the catalytic hydrogenation of carboxylic acids and their anhydrides to alcohols and/or esters in the presence of a catalyst containing a Group VIII metal, such as palladium, a metal capable of alloying with the Group VIII metal, and at least one of the metals rhenium, tungsten or molybdenum. The catalyst may be produced by I) impregnating a support with a solution or solutions of (i) at least one soluble Group VIII noble metal compound thermally decomposable/reducible to the noble metal and (ii) a soluble compound thermally decomposable/reducible to the metal of at least one metal capable of alloying with the Group VIII noble metal and removing the solvent therefrom; II) heating the composition obtain in step I) under conditions and at a temperature such that the compounds are thermally decomposed/reduced to the metals and form an alloy thereof, and III) impregnating the composition obtained in step II) with a compound of at least one of the metals rhenium, tungsten or molybdenum and removing the solvent therefrom.

U.S. Pat. No. 4,777,303 describes a process for the productions of alcohols by the hydrogenation of carboxylic acids in the presence of a catalyst that comprises a first component which is either molybdenum or tungsten and a second component which is a noble metal of Group VIII on a high surface area graphitized carbon. The catalysts may be prepared by heat treating high surface area graphitized carbon and grinding it to 16-30 mesh BSS. An aqueous solution of the first component and/or the second component is added to the carbon. The solvent is removed in a rotary evaporator and the catalyst is dried overnight in a vacuum oven. U.S. Pat. No. 4,804,791 describes another process for the production of alcohols by the hydrogenation of carboxylic acids in the presence of a catalyst prepared by heat treating a carbon support material. A noble metal and rhenium may then be co-impregnated or sequentially impregnated. U.S. Pat. No. 4,517,391 describes preparing ethanol by hydrogenating acetic acid under superatmospheric pressure and at elevated temperatures in the presence of a cobalt-containing catalyst. The catalyst is prepared in a convention manner from an appropriate mixture of metal oxides, with or without further components, e.g., phosphoric acid, and by heating this mixture for a few hours in a stream of hydrogen. During this procedure, the major part of each of the oxides is reduced to the metal.

Existing processes suffer from a variety of issues impeding commercial viability including: (i) catalysts which are possibly prohibitively expensive; (ii) catalysts without requisite selectivity to ethanol; (iii) catalysts which are nonselective for the formation of ethanol and that produce undesirable by-products.

### SUMMARY OF THE INVENTION

In a first embodiment, the invention is directed to a process for forming a catalyst for hydrogenating acetic acid and/or an ester thereof to form ethanol, the process comprising the steps of: mixing a support with at least one support modifier precursor having a metal selected from the group consisting of tungsten, molybdenum, vanadium, niobium, cobalt, tin, tantalum, and mixtures and oxides thereof to form a modified support; extruding the modified support, followed by drying and calcining, to form a pellet; and impregnating the pellet with a precious metal. The support may be selected from the group consisting of silica, alumina, titania, silica/alumina, pyrogenic silica, high purity silica, zirconia, carbon, zeolites and mixtures thereof. The metal in the support modifier precursor may be selected from the group consisting of tungsten, cobalt, tin, and mixtures or oxides thereof. In some embodiments, the metal comprises tungsten and cobalt. The packing density of the pellet is less than 0.55 g/cm³, e.g., from 0.01 to 0.55 g/cm³. The precious metal may be selected from the group consisting of rhodium, rhenium, ruthenium, platinum, palladium, osmium, iridium, gold, and mixtures thereof. The process may further comprise impregnating the pellet with active metal that is selected from the group consisting of copper, iron, vanadium, tin, cobalt, nickel, titanium, zinc, chromium, molybdenum, tungsten, lanthanum, cerium, and manganese. In some embodiments, the active metal comprises cobalt and tin. The packing density of the catalyst may be less than 1 g/cm³. In some embodiments, the process may further comprise calcining the modified support after extrusion.

In a second embodiment, the invention is directed to a process for forming a catalyst for hydrogenating acetic acid and/or an ester thereof to form ethanol, the process comprising the steps of providing a pellet comprising a support and at least one metal selected from the group consisting of tungsten, molybdenum, vanadium, niobium, cobalt, tin, tantalum, and mixtures and oxides thereof, wherein the packing density of the pellet is less than 0.55 g/cm³, and impregnating the pellet with a precious metal selected from the group consisting of rhodium, rhenium, ruthenium, platinum, palladium, osmium, iridium, gold and mixtures thereof. The pellet preferably is extruded, dried, and calcined prior to impregnating the precious metal.

In a third embodiment, the present invention is directed to a process for producing ethanol, comprising contacting a feedstock comprising acetic acid and/or an ester thereof and hydrogen in a reactor at an elevated temperature in the presence of a catalyst under conditions effective to form ethanol, wherein the catalyst is prepared by mixing a support with at least one support modifier metal selected from the group consisting of tungsten, molybdenum, vanadium, niobium, cobalt, tin, tantalum, and mixtures and oxides thereof to form a modified support; extruding, followed by drying and calcining, the modified support to form a pellet; and impregnating the pellet with a precious metal. The precious metal may be selected from the group consisting of rhodium, rhenium, ruthenium, platinum, palladium, osmium, iridium, gold and mixtures thereof. In some embodiments, the precious metal is platinum. The acetic acid may be derived from a carbonaceous material selected from the group consisting of oil, coal, natural gas and biomass. The rate of production of grams of ethanol per kilogram of catalyst per hour may at least 100 gₑₜₕₐₙₒₗ/kg_{catalyst} and the rate of production of grams of ethanol per kilogram of precious metal per hour may be at least 1000 gₑₜₕₐₙₒₗ/kg_{precious metal}. Acetic acid conversion may be at least 80% and acetic acid selectivity to ethanol may be at least 80%. Hydrogenation may be performed in a vapor phase at a temperature from 125 to 350° C., a pressure of 10 kPa to 3000 kPa, and a hydrogen to acetic acid molar ratio of at least 4:1.

### DETAILED DESCRIPTION OF THE INVENTION

### Processes for Making the Catalyst

The present invention is directed to processes for making catalysts that are suitable as hydrogenation catalysts, and to chemical processes for using the catalysts made by these processes. The catalyst comprises a precious metal and a modified support comprising a support modifier. The catalyst may further comprise at least one active metal that is either added with the support modifier or precious metal. Advantageously, the weight percent of precious metal may be maintained and the packing density of the catalyst may be lowered. Even when a relatively small weight percent of precious metal is desired, there is still a high cost associated due to the limited resources. Surprisingly and unexpectedly, extruding the support after mixing the support with a support modifier provides a pellet with lower packing density than a support that is extruded prior to impregnating the support with a support modifier. This lower packing density may lead to increased porosity and may lead to cost savings for the overall catalyst production process.

For purposes of the present invention, the term “pellet” refers to a support that comprises at support modifier and optionally, an active metal, and is extruded. Pellets may have any shape such as a sphere, cylinder, oval, etc. In some embodiments, the pellet has a packing density of less than 0.55 g/cm³, e.g., less than 0.5 g/cm³or less than 0.45 g/cm³. In terms of ranges, the pellet may have a packing density from 0.1 to 0.55 g/cm³, e.g., from 0.1 to 0.5 g/cm³, or from 0.2 to 0.45 g/cm³. In some embodiments, the pellet has a packing density that is at least 10% lower, e.g., at least 15% lower or at least 20% lower than the packing density of a support prepared by extruding the support and then modifying the support.

Without being bound by theory, it is believed that by extruding the modified support, instead of extruding the support and then modifying it, the packing density is reduced due to uniform distribution of the support modifier(s) on the support material. Thus, the extruded modified support pellet has a similar packing density to an extruded support. Conversely, when the support is extruded and then mixed with a support modifier precursor, the modified support has extra weight that leads to an increase of packing density.

The uniform distribution of the support modifier(s) on the support material may also lead to increased porosity. This increased porosity allows for impregnation of the precious metal to be achieved in fewer impregnation steps, and preferably in one impregnation step. To achieve the desired distribution and porosity, the packing density of the pellet should be less than 0.55 g/cm³. After drying and calcining the precious metal, the resulting catalyst may have a suitable packing density to be used as a hydrogenation catalyst. The packing density of the overall catalyst may be less than 1 g/cm³, e.g., less than 0.8 g/cm³or less than 0.6 g/cm³. In terms of ranges, the packing density of the overall catalyst may range from 0.2 to 1 g/cm³, e.g., from 0.3 to 0.8 g/cm³or from 0.3 to 0.6 g/cm³.

In one embodiment, the support is modified with at least one support modifier and/or the precursor thereof selected from the group consisting of tungsten, molybdenum, vanadium, niobium, cobalt, tin, tantalum and mixtures and oxides thereof, and the resulting modified support is extruded to form a pellet, and then dried and calcined. The pellet is then impregnated with a precious metal. For example, the support may be mixed with a support modifier solution comprising at least one support modifier precursor. After extrusion, drying and calcination, the resulting pellet is impregnated with a second solution comprising a precious metal precursor and optionally one or more of the at least one active metal precursors, followed by drying and calcination to form the final catalyst.

In this embodiment, the support modifier solution may comprise at least one support modifier precursor, more preferably at least two support modifier precursors. The precursors preferably are comprised of salts of the respective metals in solution, which, when heated, are converted to elemental metallic form or to a metal oxide. The at least one support modifier precursor is mixed with the support and the at least one support modifier precursor may interact with another support modifier precursor, to form one or more polymetallic crystalline species, such as cobalt tungstate. In other embodiments, the support modifiers and precursors thereof will not interact with each other and are separately deposited on the support, e.g., as discrete metal nanoparticles or as an amorphous metal mixture. Thus, the support may be modified with one support modifier precursor at the same time that it is modified with another support modifier precursor, and they may or may not interact to form one or more polymetallic crystalline species.

As indicated, in most embodiments, the support modifier preferably is added through physical mixing to form a slurry. Preferably, a support modifier precursor to the support modifier may be used. Some exemplary support modifier precursors include alkali metal oxides, alkaline earth metal oxides, Group IIB metal oxides, Group IIIB metal oxides, Group IVB metal oxides, Group VB metal oxides, Group VIB metal oxides, Group VIIB metal oxides, and/or Group VIII metal oxides, as well as preferably aqueous salts thereof. The support modifier precursor may be combined in a solution with an active metal precursor.

Although the overwhelming majority of metal oxides and polyoxoion salts are insoluble, or have a poorly defined or limited solution chemistry, the class of isopoly- and heteropolyoxoanions of the early transition elements forms an important exception. These complexes may be represented by the general formulae:

[MₘO_{y}]^{p-} Isopolyanions

[XₓMₘO_{y}]^{q-} (x≦m) Heteropolyanions

where M is selected from tungsten, molybdenum, vanadium, niobium, cobalt, tin, tantalum and mixtures thereof, in their highest (d⁰, d¹) oxidations states. Such polyoxometalate anions form a structurally distinct class of complexes based predominately, although not exclusively, upon quasi-octahedrally-coordinated metal atoms. The elements that can function as the addenda atoms, M, in heteropoly- or isopolyanions may be limited to those with both a favorable combination of ionic radius and charge and the ability to form d_{π}-p_{π} M-O bonds. There is little restriction, however, on the heteroatom, X, which may be selected from virtually any element other than the rare gases. See, e.g., M. T. Pope, Heteropoly and Isopoly Oxometalates, Springer Verlag, Berlin, 1983, 180; Chapt. 38, Comprehensive Coordination Chemistry, Vol. 3, 1028-58, Pergamon Press, Oxford, 1987, the entireties of which are incorporated herein by reference.

Polyoxometalates (POMs) and their corresponding heteropoly acids (HPAs) have several advantages making them economically and environmentally attractive. First, HPAs have a very strong approaching the superacid region, Bronsted acidity. In addition, they are efficient oxidants exhibiting fast reversible multielectron redox transformations under rather mild conditions. Solid HPAs also possess a discrete ionic structure, comprising fairly mobile basic structural units, e.g., heteropolyanions and countercations (H⁺, H₃O⁺, H₅O₂⁺, etc.), unlike zeolites and metal oxides.

In view of the foregoing, in some embodiments, the support modifier precursor comprises a POM, which preferably comprises a metal selected from the group consisting of tungsten, molybdenum, niobium, cobalt, tin, vanadium and tantalum. In some embodiments, the POM comprises a hetero-POM. A non-limiting list of suitable POMs includes phosphotungstic acid (H—PW₁₂)(H₃PW₁₂O₄₀.nH₂O), ammonium metatungstate (AMT) ((NH₄)₆H₂W₁₂O₄₀.H₂O), ammonium heptamolybdate tetrahydrate, (AHM) ((NH₄)₆Mo₇O₂₄.4H₂O), silicotungstic acid hydrate (H—SiW₁₂)(H₄SiW₁₂O₄₀.H₂O), silicomolybdic acid (H—SiMo₁₂)(H₄SiMo₁₂O₄₀.nH₂O), and phosphomolybdic acid (H—PMo₁₂)(H₃PMo₁₂O₄₀.nH₂O).

The use of POM-derived support modifiers in the catalyst compositions of the invention has now surprising and unexpectedly been shown to provide bi- or multi-functional catalyst functionality, desirably resulting in conversions for both acetic acid and byproduct esters such as ethyl acetate, thereby rendering them suitable for catalyzing mixed feeds comprising, for example, acetic acid and ethyl acetate.

Impregnation of the precious metal and the at least one active metal onto the pellet may occur simultaneously (co-impregnation) or sequentially. In simultaneous impregnation, the one or more metal precursors are mixed together and added to the pellet together followed by drying and calcination to form the final catalyst composition. With simultaneous impregnation, it may be desired to employ a dispersion agent, surfactant, or solubilizing agent, e.g., ammonium oxalate or an acid such as acetic or nitric acid, to facilitate the dispersing or solubilizing of the metal precursors in the event the precursors are incompatible with the desired solvent, e.g., water.

In sequential impregnation, the first metal precursor may be added to the pellet followed by drying and calcining, and the resulting material may then be impregnated with the remaining metal precursor each followed by an additional drying step and calcining step to form the final catalyst composition. Additional metal precursors may be added in a similar manner. Of course, combinations of sequential and simultaneous impregnation may be employed if desired.

In embodiments where the precious metal and at least one active metal are applied to the catalyst in multiple sequential impregnation steps, the catalyst may be said to comprise a plurality of “theoretical layers.” For example, where a first metal is impregnated onto a support followed by impregnation of a second metal, the resulting catalyst may be said to have a first theoretical layer comprising the first metal and a second theoretical layer comprising the second metal. As discussed above, in some aspects, more than one precursor to the support modifier may be co-impregnated onto the support in a single step such that a theoretical layer may comprise more than one metal or metal oxide. In another aspect, the same metal precursor may be impregnated in multiple sequential impregnation steps leading to the formation of multiple theoretical layers containing the same metal or metal oxide. In this context, notwithstanding the use of the term “layers,” it will be appreciated by those skilled in the art that multiple layers may or may not be formed on the catalyst support depending, for example, on the conditions employed in catalyst formation, on the amount of metal used in each step and on the specific metals employed.

The use of a solvent, such as water, glacial acetic acid, a strong acid such as hydrochloric acid, nitric acid, or sulfuric acid, or an organic solvent, is preferred in the support modification step, e.g., for impregnating a support modifier precursor onto the support. The support modifier solution comprises the solvent, preferably water, a support modifier precursor. The solution is stirred and combined with the support using, for example, incipient wetness techniques in which the support modifier precursor is added to a support having the same pore volume as the volume of the solution. Impregnation occurs by adding, optionally drop wise, a solution containing the precursors of either or both the support modifier metals to the dry support. Capillary action then draws the support modifier into the pores of the support. The thereby impregnated support can then be formed by drying, optionally under vacuum, to drive off solvents and any volatile components within the support mixture and depositing the support modifier on and/or within the support. Drying may occur, for example, at a temperature from 50° C. to 300° C., e.g., from 100° C. to 200° C. or about 120° C., for a period from 1 to 24 hours, e.g., from 3 to 15 hours or from 6 to 12 hours. The dried support may be calcined optionally with ramped heating, for example, at a temperature from 300° C. to 900° C., e.g., from 400° C. to 750° C., from 500° C. to 600° C. or at about 550° C., for a period of time from 1 to 12 hours, e.g., from 2 to 10 hours, from 4 to 8 hours or about 6 hours. Upon heating and/or the application of vacuum, the metal(s) of the precursor(s) preferably decompose into their oxide or elemental form. In some cases, the completion of removal of the solvent may not take place until the catalyst is placed into use and/or calcined, e.g., subjected to the high temperatures encountered during operation. During the calcination step, or at least during the initial phase of use of the catalyst, such compounds are converted into a catalytically active form of the metal or a catalytically active oxide thereof.

Once the support modifier precursor is mixed with the support, the modified support is extruded to have the desired size distribution, e.g., to form pellets having an average particle size in the range from 0.2 to 0.4 cm. The supports may be extruded to the desired size distribution. Any known extrusion methods to shape the support into desired size distribution can be employed. The pellet is then dried and calcined.

In one embodiment, the precious metal and, optionally, at least one active metal, are impregnated onto the pellet. A precursor of the precious metal preferably is used in the metal impregnation step, such as a water soluble compound or water dispersible compound/complex that includes the precious metal of interest. Similarly, one or more precursors to the at least one active metal may also be impregnated into pellet. Depending on the metal precursors employed, the use of a solvent, such as water, glacial acetic acid, nitric acid or an organic solvent, may be preferred to solubilize the metal precursors.

In one embodiment, separate solutions of the metal precursors are formed, which are subsequently blended prior to being impregnated on the pellet. For example, a first solution may be formed comprising a first metal precursor, and a second solution may be formed comprising a second metal precursor and optionally a third metal precursor. At least one of the first, second and optional third metal precursors preferably is a precious metal precursor, and the other(s) are preferably cobalt and/or tin precursors (which may or may not comprise precious metal precursors). Either or both solutions preferably comprise a solvent, such as water, glacial acetic acid, hydrochloric acid, nitric acid or an organic solvent.

In one exemplary embodiment, a first solution comprising a first metal halide is prepared. The first metal halide comprises a tin halide, e.g., a tin chloride such as tin (II) chloride and/or tin (IV) chloride. A second metal precursor, as a solid or as a separate solution, is combined with the first solution to form a combined solution. The second metal precursor comprises a cobalt oxalate, acetate, halide or nitrate. A second solution is also prepared comprising a precious metal precursor, such as a halide of rhodium, rhenium, ruthenium, platinum or palladium. The second solution is combined with the combined solution to form a mixed metal precursor solution. The resulting mixed metal precursor solution may then be added to the pellet, followed by drying and calcining to form the final catalyst composition as described above. The resulting catalyst may or may not be washed after the final calcination step. Due to the difficulty in solubilizing some precursors, it may be desired to reduce the pH of the first and/or second solutions, for example by employing an acid such as acetic acid, hydrochloric acid or nitric acid, e.g., 6 to 10 M HNO₃.

In another aspect, a first solution comprising a first metal oxalate is prepared, such as an oxalate of cobalt and/or tin. In this embodiment, the first solution preferably further comprises an acid such as acetic acid, hydrochloric acid, phosphoric acid or nitric acid, e.g., 6 to 10 M HNO₃. A second metal precursor, as a solid or as a separate solution, is combined with the first solution to form a combined solution. The second metal precursor, if used, preferably comprises cobalt oxalate, acetate, halide or nitrate. A second solution is also formed comprising a precious metal oxalate, for example, an oxalate of rhodium, rhenium, ruthenium, platinum or palladium, and optionally further comprises an acid such as acetic acid, hydrochloric acid, phosphoric acid or nitric acid, e.g., 6 to 10 M HNO₃. The second solution is combined with the combined solution to form a mixed metal precursor solution. The resulting mixed metal precursor solution may then be added to the pellet, followed by drying and calcining to form the final catalyst composition as described above. The resulting catalyst may or may not be washed after the final calcination step.

In one embodiment, the impregnated modified support is dried at a temperature from 100° C. to 140° C., from 110° C. to 130° C., or about 120° C., optionally from 1 to 12 hours, e.g., from 2 to 10 hours, from 4 to 8 hours or about 6 hours. The calcination temperature employed in this second step is less than the calcination temperature employed in the formation of the modified support, discussed above. The second calcination step, for example, may be conducted at a temperature that is at least 50° C., at least 100° C., at least 150° C. or at least 200° C. less than the first calcination step, i.e., the calcination step used to form the modified support. For example, the impregnated catalyst may be calcined at a temperature from 200° C. to 500° C., from 300° C. to 400° C., or about 350° C., optionally for a period from 1 to 12 hours, e.g., from 2 to 10 hours, from 4 to 8 hours or about 6 hours.

In one embodiment, ammonium oxalate is used to solubilize at least one of the metal precursors, e.g., a tin precursor, as described in U.S. Pat. No. 8,211,821, the entirety of which is incorporated herein by reference. In this aspect, a solution of the second metal precursor may be made in the presence of ammonium oxalate as solubilizing agent, and the precious metal precursor may be added thereto, optionally as a solid or a separate solution. If used, the third metal precursor may be combined with the solution comprising the first precursor and tin oxalate precursor, or may be combined with the second metal precursor, optionally as a solid or a separate solution, prior to addition of the first metal precursor. In other embodiments, an acid such as acetic acid, hydrochloric acid or nitric acid may be substituted for the ammonium oxalate to facilitate solubilizing of the tin oxalate. The resulting mixed metal precursor solution may then be added to the pellet followed by drying and calcining to form the final catalyst composition as described above.

The specific precursors used in the various embodiments of the invention may vary widely. Suitable metal precursors may include, for example, metal halides, amine solubilized metal hydroxides, metal nitrates or metal oxalates. For example, suitable compounds for platinum precursors and palladium precursors include chloroplatinic acid, ammonium chloroplatinate, amine solubilized platinum hydroxide, platinum nitrate, platinum tetra ammonium nitrate, platinum chloride, platinum oxalate, palladium nitrate, palladium tetra ammonium nitrate, palladium chloride, palladium oxalate, sodium palladium chloride, sodium platinum chloride, and platinum ammonium nitrate, Pt(NH₃)₄(NO₄)₂. Generally, both from the point of view of economics and environmental aspects, aqueous solutions of soluble compounds of platinum and palladium are preferred. In one embodiment, the precious metal precursor is not a metal halide and is substantially free of metal halides, while in other embodiments, as described above, the precious metal precursor is a halide.

In another example, cobalt and/tin are co-impregnated with the tungsten precursor on the support and may form a mixed oxide with WO₃, e.g., cobalt tungstate, followed by extruding, drying, and calcining to form a pellet. The resulting pellet may be impregnated, preferably in a single impregnation step or multiple impregnation steps, with one or more of the precious metals and optionally at least one active metal, followed by a second drying and calcination step. In this manner, when the at least one active metal comprises cobalt, cobalt tungstate may be formed on the pellet. Again, the temperature of the second calcining step preferably is less than the temperature of the first calcining step.

### Catalyst Composition

The present invention is also directed to catalyst compositions that preferably are suitable as hydrogenation catalysts and to chemical processes employing such catalysts. It has now been discovered that such catalysts are particularly effective as multifunctional hydrogenation catalysts capable of converting both carboxylic acids, such as acetic acid, and esters thereof, e.g., ethyl acetate, to their corresponding alcohol(s), e.g., ethanol, under hydrogenation conditions. The catalysts preferably comprise a precious metal and at least one active metal on a pellet that is extruded in accordance with embodiments of the present invention, and may be suitable in catalyzing the hydrogenation of a carboxylic acid, e.g., acetic acid, and/or esters thereof, e.g., ethyl acetate, to the corresponding alcohol, e.g., ethanol.

In one embodiment, the inventive catalyst comprises a precious metal and at least one active metal on a pellet. The support is modified as described above with a support modifier comprising a metal selected from tungsten, molybdenum, vanadium, niobium, cobalt, tin and tantalum, and extruded to form a pellet.

The catalysts of the invention preferably include at least one precious metal impregnated on the catalyst support. The precious metal may be selected, for example, from rhodium, rhenium, ruthenium, platinum, palladium, osmium, iridium, gold and mixtures thereof. Preferred precious metals for the catalysts of the invention include palladium, platinum, and rhodium. The precious metal preferably is catalytically active in the hydrogenation of a carboxylic acid and/or its ester to the corresponding alcohol(s). The precious metal may be in elemental form or in molecular form, e.g., an oxide of the precious metal. The catalyst comprises such precious metals in an amount less than 5 wt. %, e.g., less than 3 wt. %, less than 2 wt. %, less than 1 wt. % or less than 0.5 wt. %. In terms of ranges, the catalyst may comprise the precious metal in an amount from 0.05 to 10 wt. %, e.g. from 0.1 to 5 wt. %, or from 0.1 to 3 wt. %, based on the total weight of the catalyst. In some embodiments, the metal loading of the precious metal may be less than the metal loadings of the at least one active metal. As discussed above, by extruding the support after it has been modified with the at least one support modifier, the packing density of the pellet is decreased and the porosity of the pellet is increased.

In addition to the precious metal, the catalyst may include at least one active metal disposed on the pellet. In some embodiments, the at least one active metal comprises cobalt and tin. Cobalt and tin may also be used as support modifiers, preferably in combination with tungsten. Without being bound by theory, the cobalt and tin may disperse the tungsten or oxide thereof on the support. Cobalt and tin are part of the modified support when they are impregnated and calcined on the support prior to the impregnation or introduction of the precious metal to the modified support, e.g., as support modifiers.

As used herein, active metals refer to catalytically active metals that improve the conversion, selectivity and/or productivity of the catalyst and may include precious or non-precious active metals. Thus, a catalyst comprising a precious metal and at least one active metal may include: (i) one (or more) precious metals and one (or more) non-precious active metals, or (ii) may comprise two (or more) precious metals. Thus, precious metals are included herein as exemplary active metals. Further, it should be understood that use of the term “active metal” to refer to some metals in the catalysts of the invention is not meant to suggest that the precious metal that is also included in the inventive catalysts is not catalytically active.

In one embodiment, the at least one active metal included in the catalyst is selected from the group consisting of copper, iron, cobalt, vanadium, nickel, titanium, zinc, chromium, molybdenum, tungsten, tin, lanthanum, cerium, manganese, any of the aforementioned precious metals, in particular rhenium, ruthenium, and gold, and combinations thereof. Preferably, however, the at least one active metal does not include any precious metals, and thus include copper, iron, cobalt, vanadium, nickel, titanium, zinc, chromium, molybdenum, tungsten, tin, lanthanum, cerium, manganese, and combinations thereof. More preferably, the at least one active metal is selected from the group consisting of copper, iron, cobalt, nickel, chromium, molybdenum, tungsten and tin, and more preferably the at least one active metal comprises cobalt and tin. The at least one active metal may be in elemental form or in molecular form, e.g., an oxide of the active metal, or a combination thereof.

The total weight of all the active metals, including the aforementioned precious metal, present in the catalyst preferably is from 0.1 to 25 wt. %, e.g., from 0.5 to 15 wt. %, or from 1.0 to 10 wt. %. In one embodiment, the catalyst may comprise from cobalt in an amount from 0.5 to 20 wt. %, e.g., preferably from 4.1 to 20 wt. %, and tin in an amount from 0.5 to 20 wt. %, e.g., preferably from 0.5 to 3.5 wt. %. In other embodiments, when the pellet comprises cobalt and tungsten, the catalyst may comprise tin as the at least one active metal and the tin may be present from 0.5 to 20 wt. %, e.g., from 0.5 to 3.5 wt. %.

The at least one active metal for purposes of the present invention is disposed on the modified support, e.g., pellet, and may be part of the modified support. In embodiments where the support modifier is cobalt, tin, tungsten, or molybdenum, the total weight of the active metal may include the combined weight of the active metal and the support modifier. Thus, for example, the modified support may comprise from 0.1 to 15 wt. %, e.g. from 0.5 to 10 wt. %, of cobalt, tin, tungsten, or molybdenum and the at least one active metal disposed on the modified support may be present in an amount from 0.1 to 15 wt. %, e.g., from 0.5 to 10 wt. %, provided that the total metal loading of the at least one active metal is less than 25 wt. %. For purposes of the present specification, unless otherwise indicated, weight percent is based on the total weight the catalyst including metal and support.

In some embodiments, the catalyst contains at least two active metals in addition to the precious metal. The at least two active metals may be selected from any of the active metals identified above, so long as they are not the same as the precious metal or each other. Additional active metals may also be used in some embodiments. Thus, in some embodiments, there may be multiple active metals on the support in addition to the precious metal.

Preferred bimetallic (precious metal+active metal) combinations for adding to the pellet in some exemplary catalyst compositions include platinum/tin, platinum/ruthenium, platinum/rhenium, platinum/cobalt, platinum/nickel, palladium/ruthenium, palladium/rhenium, palladium/cobalt, palladium/copper, palladium/nickel, ruthenium/cobalt, gold/palladium, ruthenium/rhenium, ruthenium/iron, rhodium/iron, rhodium/cobalt, rhodium/nickel and rhodium/tin. In some embodiments, the catalyst comprises three metals impregnated on a pellet, e.g., one precious metal and two active metals. Exemplary tertiary combinations may include palladium/rhenium/tin, palladium/rhenium/cobalt, palladium/rhenium/nickel, palladium/cobalt/tin, platinum/tin/palladium, platinum/tin/rhodium, platinum/tin/gold, platinum/tin/iridium, platinum/cobalt/tin, platinum/tin/copper, platinum/tin/chromium, platinum/tin/zinc, platinum/tin/nickel, rhodium/nickel/tin, rhodium/cobalt/tin and rhodium/iron/tin. In one preferred embodiment, the tertiary combination comprises cobalt or tin or both cobalt and tin. In some embodiments, the catalyst may comprise more than three metals on the support.

When the catalyst comprises a precious metal and one active metal on a support, the active metal is present in an amount from 0.1 to 20 wt. %, e.g., from 0.1 to 10 wt. %, or from 0.1 to 7.5 wt. %. When the catalyst comprises two or more active metals in addition to the precious metal, e.g., a first active metal and a second active metal, the first active metal may be present in the catalyst in an amount from 0.05 to 20 wt. %, e.g. from 0.1 to 10 wt. %, or from 0.5 to 5 wt. %. The second active metal may be present in an amount from 0.05 to 20 wt. %, e.g., from 0.1 to 10 wt. %, or from 0.5 to 7.5 wt. %. If the catalyst further comprises a third active metal, the third active metal may be present in an amount from 0.05 to 20 wt. %, e.g., from 0.05 to 10 wt. %, or from 0.05 to 7.5 wt. %. When the second or third active metal is cobalt, in one embodiment, the metal loading may be from 4.1 to 20 wt. %, e.g., from 4.1 to 10 wt. % or from 4.1 to 7.5 wt. %. The active metals may be alloyed with one another or may comprise a non-alloyed metal solution, a metal mixture or be present as one or more metal oxides.

The preferred metal ratios may vary somewhat depending on the active metals used in the catalyst. In some embodiments, the mole ratio of the precious metal to the at least one active metal is from 10:1 to 1:10, e.g., from 4:1 to 1:4, from 2:1 to 1:2 or from 1.5:1 to 1:1.5. In another embodiment, the precious metal may be present in an amount from 0.1 to 5 wt. %, the first active metal in an amount from 0.5 to 20 wt. % and the second active metal in an amount from 0.5 to 20 wt. %, based on the total weight of the catalyst. In another embodiment, the precious metal is present in an amount from 0.1 to 5 wt. %, the first active metal in an amount from 0.5 to 15 wt. % and the second active metal in an amount from 0.5 to 15 wt. %.

In one embodiment, the first and second active metals are present as cobalt and tin, and, when added to the catalyst together and calcined together, are present at a cobalt to tin molar ratio from 6:1 to 1:6 or from 3:1 to 1:3. The cobalt and tin may be present in substantially equimolar amounts, when added to the catalyst together and calcined together. In another embodiment, when cobalt is added to the support initially and calcined as part of the modified support and tin is subsequently added with the precious metal to the pellet, it is preferred to have a cobalt to tin molar that is greater than 4:1, e.g., greater than 6:1 or greater than 11:1. Without being bound by theory, the excess cobalt, based on molar amount relative to tin, may improve the multifunctionality of the catalyst.

In one embodiment, the support may be an inorganic oxide. The support, prior to modification, may be selected from the group consisting of silica, alumina, titania, silica/alumina, pyrogenic silica, high purity silica, zirconia, carbon (e.g., carbon black or activated carbon), zeolites and mixtures thereof. Preferably, the support comprises a silicaceous support such as silica, pyrogenic silica, or high purity silica. In one embodiment the silicaceous support is substantially free of alkaline earth metals, such as magnesium and calcium. In preferred embodiments, the support is present in an amount from 25 wt. % to 99 wt. %, e.g., from 30 wt. % to 98 wt. % or from 35 wt. % to 95 wt. %, based on the total weight of the catalyst.

In preferred embodiments, the support comprises a silicaceous support, e.g., silica, having a surface area of at least 50 m²/g, e.g., at least 100 m²/g, or at least 150 m²/g. In terms of ranges, the silicaceous support preferably has a surface area from 50 to 600 m²/g, e.g., from 100 to 500 m²/g or from 100 to 300 m²/g. High surface area silica, as used throughout the application, refers to silica having a surface area of at least 250 m²/g. For purposes of the present specification, surface area refers to BET nitrogen surface area, meaning the surface area as determined by ASTM D6556-04, the entirety of which is incorporated herein by reference.

The preferred silicaceous support also preferably has an average pore diameter from 5 to 100 nm, e.g., from 5 to 30 nm, from 5 to 25 nm or from 5 to 10 nm, as determined by mercury intrusion porosimetry, and an average pore volume from 0.5 to 2.0 cm³/g, e.g., from 0.7 to 1.5 cm³/g or from 0.8 to 1.3 cm³/g, as determined by mercury intrusion porosimetry.

The silicaceous support prior to impregnation and extrusion has a packing density from 0.1 to 0.5 g/cm³, e.g., from 0.2 to 0.45 g/cm³ or from 0.3 to 0.43 g/cm³. In terms of size, the silica support material preferably has an average particle size, meaning the average diameter for spherical particles or average longest dimension for non-spherical particles, from 0.01 to 1.0 cm, e.g., from 0.1 to 0.7 cm or from 0.2 to 0.5 cm. Since the precious metal and the at least one active metal that are disposed on the pellet are generally in the form of very small metal (or metal oxide) particles or crystallites relative to the size of the pellet, these metals should not substantially impact the size of the overall catalyst particles.

The support preferably comprises at least one support modifier. A support modifier may adjust the acidity of the support. In one embodiment, the at least one support modifier is selected from the group consisting of tungsten, molybdenum, vanadium, niobium, cobalt, tin and tantalum. The metal for the support modifier may be an oxide thereof. In one embodiment, the support modifiers are present in an amount from 0.1 wt. % to 50 wt. %, e.g., from 0.2 wt. % to 25 wt. %, from 0.5 wt. % to 20 wt. %, or from 1 wt. % to 15 wt. %, based on the total weight of the catalyst. When the support modifier comprises tungsten, molybdenum, or vanadium, the support modifier may be present in an amount from 0.1 to 40 wt. %, e.g., from 0.1 to 30 wt. % or from 0.1 to 20 wt. %, based on the total weight of the catalyst.

As indicated, the support modifiers may adjust the acidity of the support. For example, the acid sites, e.g., Brønsted acid sites or Lewis acid sites, on the support may be adjusted by the support modifier to favor selectivity to ethanol during the hydrogenation of acetic acid and/or esters thereof. The acidity of the support may be adjusted by optimizing surface acidity of the support. The support may also be adjusted by having the support modifier change the pKa of the support. Unless the context indicates otherwise, the acidity of a surface or the number of acid sites thereupon may be determined by the technique described in F. Delannay, Ed., “Characterization of Heterogeneous Catalysts”; Chapter III: Measurement of Acidity of Surfaces, p. 370-404; Marcel Dekker, Inc., N.Y. 1984, the entirety of which is incorporated herein by reference. In general, the surface acidity of the support may be adjusted based on the composition of the feed stream being sent to the hydrogenation process in order to maximize alcohol production, e.g., ethanol production.

In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIII metals, aluminum oxides, and mixtures thereof. In one embodiment, the support modifier comprises metal selected from the group consisting of tungsten, molybdenum, vanadium, niobium, and tantalum.

In one embodiment, the acidic modifier may also include those selected from the group consisting of WO₃, MoO₃, V₂O₅, VO₂, V₂O₃, Nb₂O₅, Ta₂O₅, FeO, Fe₃O₄, Fe₂O₃, Cr₂O₃, MnO₂, CoO, Co₂O₃, and Bi₂O₃. Reduced tungsten oxides or molybdenum oxides may also be employed, such as, for example, one or more of W₂₀O₅₈, WO₂, W₄₉O₁₁₉, W₅₀O₁₄₈, W₁₈O₄₉, Mo₉O₂₆, Mo₈O₂₃, Mo₅O₁₄, Mo₁₇O₄₇, Mo₄O₁₁, or MoO₂. In one embodiment, the tungsten oxide may be cubic tungsten oxide (H_{0.5}WO₃). It has now surprisingly and unexpectedly been discovered that the use of such metal oxide support modifiers in combination with a precious metal and at least one active metal may result in catalysts having multifunctionality, and which may be suitable for converting a carboxylic acid, such as acetic acid, as well as corresponding esters thereof, e.g., ethyl acetate, to one or more hydrogenation products, such as ethanol, under hydrogenation conditions.

In other embodiments, the acidic support modifiers include those selected from the group consisting of TiO₂, ZrO₂, Nb₂O₅, Ta₂O₅, Al₂O₃, B₂O₃, P₂O₅, and Sb₂O₃. Acidic support modifiers include those selected from the group consisting of TiO₂, ZrO₂, Nb₂O₅, Ta₂O₅, and Al₂O₃.

In some embodiments, the acidic support modifier comprises a mixed metal oxide comprising at least one of the support modifiers and an oxide anion of a Group IVB, VB, VIB, VIII metal, such as tungsten, molybdenum, vanadium, niobium, cobalt, tin or tantalum. The oxide anion, for example, may be in the form of a tungstate, molybdate, vanadate, or niobate. Exemplary mixed metal oxides include cobalt tungstate, copper tungstate, iron tungstate, zirconium tungstate, manganese tungstate, cobalt molybdate, copper molybdate, iron molybdate, zirconium molybdate, manganese molybdate, cobalt vanadate, copper vanadate, iron vanadate, zirconium vanadate, manganese vanadate, cobalt niobate, copper niobate, iron niobate, zirconium niobate, manganese niobate, cobalt tantalate, copper tantalate, iron tantalate, zirconium tantalate, and/or manganese tantalate. In one embodiment, the catalyst does not comprise tin tungstate and is substantially free of tin tungstate. It has now been discovered that catalysts containing such mixed metal support modifiers may provide the desired degree of multifunctionality at increased conversion, e.g., increased ester conversion, and with reduced byproduct formation, e.g., reduced diethyl ether formation.

In one embodiment, the catalyst comprises from 0.25 to 1.25 wt. % platinum, from 1 to 10 wt. % cobalt, and from 1 to 10 wt. % tin on a silica or a silica-alumina modified support pellet. The support may comprise from 5 to 15 wt. % acidic support modifiers, such as WO₃, V₂O₅ and/or MoO₃. In one embodiment, the acidic modifier may comprise cobalt tungstate, e.g., in an amount from 0.1 to 20 wt. %, or from 5 to 15 wt. %.

In another embodiment, the catalyst comprises from 0.25 to 2.5 wt. % platinum, from 1 to 10 wt. % cobalt, and from 1 to 10 wt. % tin on a silica or a silica-alumina modified support pellet. The support may comprise from 5 to 20 wt. % acidic support modifiers, such as WO₃, V₂O₅ and/or MoO₃. In one embodiment, the acidic modifier may comprise cobalt tungstate, e.g., in an amount from 0.1 to 20 wt. %, or from 5 to 15 wt. %.

In some embodiments, the modified support comprises at least one additional support modifier in addition to the one or more acidic modifiers. The modified support may, for example, comprise at least one active metal selected from copper, iron, cobalt, vanadium, nickel, titanium, zinc, chromium, molybdenum, tungsten, tin, lanthanum, cerium, and manganese. Preferably, the support comprises a support modifier metal selected from the group consisting of tungsten, molybdenum, vanadium, niobium, cobalt and tin and tantalum. In this aspect, the final catalyst composition comprises a precious metal, and at least one active metal disposed on the modified support, prior to extrusion. In a preferred embodiment, at least one of the support modifiers in the modified support is the same as at least one of the active metals disposed on the pellet. For example, the catalyst may comprise a support modified with cobalt, tin and tungsten (optionally as WO₃, H_{0.5}WO₃, HWO₄, and/or as cobalt tungstate). In this example, the catalyst further comprises a precious metal, e.g., palladium, platinum or rhodium, and at least one active metal, e.g., cobalt and/or tin, disposed on the modified support.

Without being by bound theory, it is believed that the presence of tin tungstate on the modified support or catalyst tends to decrease catalytic activity in the conversion of acetic acid to ethanol. When used on the modified support, tin does contribute to improved catalytic activity and catalyst lifetime. However, when tin is present with tungsten, the undesirable tin tungstate species may form. To prevent the formation of tin tungstate, it has been found the use of cobalt may inhibit the formation of tin tungstate. This allows the preferential formation of cobalt tungstate over tin tungstate. In addition, this allows the use of tin on the modified support to thus maintain sufficient catalyst activity and catalyst lifetime. In one embodiment, the modified support comprises cobalt tungstate and tin, but the modified support is substantially free of tin tungstate.

Use of Catalyst to Hydrogenate Acetic Acid and/or Ethyl Acetate

One advantage of catalysts of the present invention is the stability or activity of the catalyst for producing ethanol. Accordingly, it can be appreciated that the catalysts of the present invention are fully capable of being used in commercial scale industrial applications for hydrogenation of acetic acid, particularly in the production of ethanol.

After the washing, drying and calcining of the catalyst is completed, the catalyst may be reduced in order to activate it. Reduction is carried out in the presence of a reducing gas, preferably hydrogen. The reducing gas is optionally continuously passed over the catalyst at an initial ambient temperature that is increased up to 400° C. In one embodiment, the reduction is carried out after the catalyst has been loaded into the reaction vessel where the hydrogenation will be carried out.

In one embodiment the invention is to a process for producing ethanol by hydrogenating a feed stream comprising compounds selected from acetic acid, ethyl acetate and mixtures thereof in the presence of any of the above-described catalysts. One particular preferred reaction is to make ethanol from acetic acid. The hydrogenation reaction may be represented as follows:

HOAc+2H₂ [symbol] EtOH+H₂O

In some embodiments, the catalyst may be characterized as a bifunctional catalyst in that it effectively catalyzes the hydrogenation of acetic acid to ethanol as well as the conversion of ethyl acetate to one or more products, preferably ethanol.

The raw materials, acetic acid and hydrogen, fed to the reactor used in connection with the process of this invention may be derived from any suitable source including natural gas, petroleum, coal, biomass, and so forth. As examples, acetic acid may be produced via methanol carbonylation, acetaldehyde oxidation, ethane oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from other carbon sources have drawn increasing interest. In particular, when petroleum is relatively expensive, it may become advantageous to produce acetic acid from synthesis gas (“syngas”) that is derived from other available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

Biomass-derived syngas has a detectable ¹⁴C isotope content as compared to fossil fuels such as coal or natural gas. An equilibrium forms in the Earth's atmosphere between constant new formation and constant degradation, and so the proportion of the ¹⁴C nuclei in the carbon in the atmosphere on Earth is constant over long periods. The same distribution ratio n¹⁴C:n¹²C ratio is established in living organisms as is present in the surrounding atmosphere, which stops at death and ¹⁴C decomposes at a half life of about 6000 years. Methanol, acetic acid and/or ethanol formed from biomass-derived syngas would be expected to have a ¹⁴C content that is substantially similar to living organisms. For example, the ¹⁴C:¹²C ratio of the methanol, acetic acid and/or ethanol may be from one half to about 1 of the ¹⁴C:¹²C ratio for living organisms. In other embodiments, the syngas, methanol, acetic acid and/or ethanol described herein are derived wholly from fossil fuels, i.e. carbon sources produced over 60,000 years ago, may have no detectable ¹⁴C content.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. The microorganism employed in the fermentation process may be Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally, in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. No. 6,509,180, and U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. Another biomass source is black liquor, which is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syngas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into syngas, and U.S. Pat. No. 6,685,754, which discloses a method for the production of a hydrogen-containing gas composition, such as a syngas including hydrogen and carbon monoxide, are incorporated herein by reference in their entireties.

The acetic acid fed to the hydrogenation reactor may also comprise other carboxylic acids and anhydrides, as well as aldehyde and/or ketones, such as acetaldehyde and acetone. Preferably, the feed stream comprises acetic acid and ethyl acetate. A suitable acetic acid feed stream comprises one or more of the compounds selected from the group consisting of acetic acid, acetic anhydride, acetaldehyde, ethyl acetate, diethyl acetal, diethyl ether, and mixtures thereof. These other compounds may also be hydrogenated in the processes of the present invention. In some embodiments, the presence of carboxylic acids, such as propanoic acid or its aldehyde, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the hydrogenation reactor without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

The reactor, in some embodiments, may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed as the reactor, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation in the reactor may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa, or from 100 kPa to 2000 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 18:1 to 2:1. Most preferably, the molar ratio of hydrogen to acetic acid is greater than 2:1, e.g., greater than 4:1 or greater than 8:1. For a mixed feed stream, the molar ratio of hydrogen to ethyl acetate may be greater than 5:1, e.g., greater than 10:1 or greater than 15:1.

Contact or residence time can also vary widely, depending upon such variables as amount of feed stream (acetic acid and/or ethyl acetate), catalyst, reactor, temperature, and pressure. Typical contact times range from a fraction of a second to more than several hours when a catalyst system other than a fixed bed is used, with preferred contact times, at least for vapor phase reactions, from 0.1 to 100 seconds, e.g., from 0.3 to 80 seconds or from 0.4 to 30 seconds.

In particular, by employing the catalysts of the invention, the hydrogenation of acetic acid and/or ethyl acetate may achieve favorable conversion and favorable selectivity and productivity to ethanol in the reactor. For purposes of the present invention, the term “conversion” refers to the amount of acetic acid or ethyl acetate, whichever is specified, in the feed that is converted to a compound other than acetic acid or ethyl acetate, respectively. Conversion is expressed as a percentage based on acetic acid or ethyl acetate in the feed. The acetic acid conversion may be at least 80%, e.g., at least 90%, at least 95% or at least 99%.

During the hydrogenation of acetic acid, ethyl acetate may be produced as a byproduct. Without consuming any ethyl acetate from the mixed vapor phase reactants, the conversion of ethyl acetate would be deemed negative. Some of the catalysts described herein are monofunctional in nature and are effective for converting acetic acid to ethanol, but not for converting ethyl acetate. The use of monofunctional catalysts may result in the undesirable build up of ethyl acetate in the system, particularly for systems employing one or more recycle streams that contain ethyl acetate to the reactor.

The preferred catalysts of the invention, however, are multifunctional in that they effectively catalyze the conversion of acetic acid to ethanol as well as the conversion of an alkyl acetate, such as ethyl acetate, to one or more products other than that alkyl acetate. The multifunctional catalyst is preferably effective for consuming ethyl acetate at a rate sufficiently great so as to at least offset the rate of ethyl acetate production, thereby resulting in a non-negative ethyl acetate conversion, i.e., no net increase in ethyl acetate is realized. The use of such catalysts may result, for example, in an ethyl acetate conversion that is effectively 0% or that is greater than 0%. In some embodiments, the catalysts of the invention are effective in providing ethyl acetate conversions of at least 0%, e.g., at least 5%, at least 10%, at least 15%, at least 20%, or at least 35%.

In continuous processes, the ethyl acetate being added (e.g., recycled) to the hydrogenation reactor and ethyl acetate leaving the reactor in the crude product preferably approaches a certain level after the process reaches equilibrium. The use of a multifunctional catalyst that catalyzes the conversion of ethyl acetate as well as acetic acid results in a lower amount of ethyl acetate added to the reactor and less ethyl acetate produced relative to monofunctional catalysts. In preferred embodiments, the concentration of ethyl acetate in the mixed feed and crude product is less than 40 wt. %, less than 25 wt. % or less than 15 wt. %, after equilibrium has been achieved. In preferred embodiments, the process forms a crude product comprising ethanol and ethyl acetate, and the crude product has an ethyl acetate steady state concentration from 0.1 to 40 wt. %, e.g., from 0.1 to 20 wt. % or from 0.1 to 15 wt. %.

Although catalysts that have high acetic acid conversions are desirable, such as at least 60%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid and/or ethyl acetate. It should be understood that each compound converted from acetic acid and/or ethyl acetate has an independent selectivity and that selectivity is independent of conversion. For example, if 60 mole % of the converted acetic acid is converted to ethanol, we refer to the ethanol selectivity as 60%. For purposes of the present invention, the total selectivity is based on the combined converted acetic acid and ethyl acetate. Preferably, total selectivity to ethanol is at least 80%, e.g., at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 100 grams of ethanol per kilogram of catalyst (gₑₜₕₐₙₒₗ/kg_{catalyst}) per hour, e.g., at least 400 gₑₜₕₐₙₒₗ/kg_{catalyst} per hour or at least 600 gₑₜₕₐₙₒₗ/kg_{catalyst} per hour, is preferred. In terms of ranges, the productivity preferably is from 100 to 3,000 gₑₜₕₐₙₒₗ/kg_{catalyst} per hour, e.g., from 400 to 2,500 gₑₜₕₐₙₒₗ/kg_{catalyst} per hour or from 600 to 2,000 gₑₜₕₐₙₒₗ/kg_{catalyst} per hour.

In some embodiments, due to the decreased packing density of the pellet and the resulting lower amount, on a mass basis, of precious metal required, a productivity of at least 1000 grams of ethanol per kilogram of precious metal (gₑₜₕₐₙₒₗ/kg_{precious metal}) per hour is preferred, e.g., at least 2000 gₑₜₕₐₙₒₗ/kg_{precious metal} per hour, or at least 4000 gₑₜₕₐₙₒₗ/kg_{precious metal} per hour. In terms of ranges, the productivity preferably is from 1000 to 4,000 gₑₜₕₐₙₒₗ/kg_{precious metal} per hour, e.g., from 1000 to 3000 gₑₜₕₐₙₒₗ/kg_{precious metal} per hour or from 1000 to 2000 gₑₜₕₐₙₒₗ/kg_{precious metal} per hour.

In various embodiments of the present invention, the crude ethanol product produced by the reactor, before any subsequent processing, such as purification and separation, will typically comprise unreacted acetic acid, ethanol and water. Exemplary compositional ranges for the crude ethanol product are provided in Table 1. The “others” identified in Table 1 may include, for example, esters, ethers, aldehydes, ketones, alkanes, and carbon dioxide.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 72 | 15 to 72 | 15 to 70 | 25 to 65 |
| Acetic Acid | 0 to 90 | 0 to 50 | 0 to 35 | 0 to 15 |
| Water | 5 to 40 | 5 to 30 | 10 to 30 | 10 to 26 |
| Ethyl Acetate | 0 to 30 | 1 to 25 | 3 to 20 | 5 to 18 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product may comprise acetic acid in an amount less than 20 wt. %, e.g., of less than 15 wt. %, less than 10 wt. % or less than 5 wt. %. In terms of ranges, the acetic acid concentration of Table 1 may range from 0.1 wt. % to 20 wt. %, e.g., 0.1 wt. % to 15 wt. %, from 0.1 wt. % to 10 wt. % or from 0.1 wt. % to 5 wt. %. In some embodiments where the crude ethanol product has a lower amount of acetic acid, the conversion of acetic acid is preferably greater than 75%, e.g., greater than 85% or greater than 90%. In addition, the selectivity to ethanol may also be preferably high, and is greater than 75%, e.g., greater than 85% or greater than 90%.

An ethanol product may be recovered from the crude ethanol product produced by the reactor using the catalyst of the present invention may be recovered using several different techniques.

The ethanol product may be an industrial grade ethanol comprising from 75 to 96 wt. % ethanol, e.g., from 80 to 96 wt. % or from 85 to 96 wt. % ethanol, based on the total weight of the ethanol product. The industrial grade ethanol may have a water concentration of less than 12 wt. % water, e.g., less than 8 wt. % or less than 3 wt. %. In some embodiments, when further water separation is used, the ethanol product preferably contains ethanol in an amount that is greater than 96 wt. %, e.g., greater than 98 wt. % or greater than 99.5 wt. %. The ethanol product having further water separation preferably comprises less than 3 wt. % water, e.g., less than 2 wt. % or less than 0.5 wt. %.

The finished ethanol composition produced by the embodiments of the present invention may be used in a variety of applications including fuels, solvents, chemical feedstocks, pharmaceutical products, cleansers, sanitizers, hydrogen transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, ethyl benzene, aldehydes, butadiene, and higher alcohols, especially butanol. In the production of ethyl acetate, the finished ethanol composition may be esterified with acetic acid. In another application, the finished ethanol composition may be dehydrated to produce ethylene. Any known dehydration catalyst, such as zeolite catalysts or phosphotungstic acid catalysts, can be employed to dehydrate ethanol, as described in U.S. Pub. Nos. 2010/0030002 and 2010/0030001 and WO2010146332, the entire contents and disclosures of which are hereby incorporated by reference.

The following examples describe the catalyst and process of this invention.

### EXAMPLES

### Example 1

A silica support was modified with tungsten oxide and cobalt to provide a modified support with 12 wt. % tungsten oxide and 7.5 wt. % cobalt. The modified support was then extruded, dried, and calcined to form a pellet. The pellet had a packing density of 0.454 g/cm³.

### Comparative Example A

A silica support was extruded. The extruded support was then modified with tungsten oxide and cobalt as above. The modified support was then dried and calcined. The modified support that was extruded prior to modification had a packing density of 0.568 g/cm³.

### Example 2

A silica support was modified with tungsten oxide provide a modified support with 12 wt. % tungsten oxide. The modified support was then extruded, dried and calcined. The extruded modified support had a packing density of 0.362 g/cm³.

### Example 3

A silica support was prepared as in Example 2. The extruded modified support had a packing density of 0.364 g/cm³.

### Example 4

A silica support was modified with tungsten oxide to provide a modified support with 17 wt. % tungsten oxide. The modified support was then extruded, dried and calcined. The extruded modified support had a packing density of 0.454 g/cm³.

### Example 5

A silica support was prepared with tungsten oxide, cobalt and tin provide a modified support with 12 wt. % tungsten oxide, 3.75 wt. % cobalt and 3.25 wt. % tin. The modified support was then extruded, dried and calcined. The extruded modified support had a packing density of 0.420 g/cm³.

When testing Example 1 and Comparative Example A, extruding the support after modification from Example 1 resulted in a 20% decrease in packing density.

While the invention has been described in detail, modifications within the spirit and scope of the invention will be readily apparent to those skilled in the art. All publications and references discussed above are incorporated herein by reference. In addition, it should be understood that aspects of the invention and portions of various embodiments and various features recited may be combined or interchanged either in whole or in part. In the foregoing descriptions of the various embodiments, those embodiments which refer to another embodiment may be appropriately combined with other embodiments as will be appreciated by one skilled in the art. Furthermore, those skilled in the art will appreciate that the foregoing description is by way of example only, and is not intended to limit the invention.

We claim:

1. A process for forming a catalyst for hydrogenating acetic acid and/or an ester thereof to form ethanol, the process comprising the steps of:
(a) mixing a support with at least one support modifier precursor having a metal selected from the group consisting of tungsten, molybdenum, vanadium, niobium, cobalt, tin, tantalum, and mixtures thereof to form a modified support;
(b) extruding the modified support to form a pellet; and
(c) impregnating the pellet with a precious metal.

2. The process of claim 1, wherein the support is selected from the group consisting of silica, alumina, titania, silica/alumina, pyrogenic silica, high purity silica, zirconia, carbon, zeolites and mixtures thereof.

3. The process of claim 1, wherein the metal is selected from the group consisting of tungsten, cobalt, tin, and mixtures or oxides thereof.

4. The process of claim 1, wherein the metal comprises tungsten and cobalt.

5. The process of claim 1, wherein the precious metal is selected from the group consisting of rhodium, rhenium, ruthenium, platinum, palladium, osmium, iridium, gold and mixtures thereof.

6. The process of claim 1, further comprising impregnating the pellet with at least one active metal that is selected from the group consisting of copper, iron, vanadium, tin, cobalt, nickel, titanium, zinc, chromium, molybdenum, tungsten, lanthanum, cerium, and manganese.

7. The process of claim 6, wherein the at least one active metal comprises cobalt and tin.

8. The process of claim 1, wherein the packing density of the catalyst is less than 1 g/cm³.

9. The process of claim 1, wherein the packing density of the pellet is less than 0.55 g/cm³.

10. The process of claim 1, wherein the packing density of the pellet is from 0.01 to 0.55 g/cm³.

11. The process of claim 1, further comprising calcining the modified support after extrusion.

12. A process for producing ethanol, comprising contacting a feedstock comprising acetic acid and/or an ester thereof and hydrogen in a reactor at an elevated temperature in the presence of a catalyst made by the process of claim 1, under conditions effective to form ethanol.

13. The process of claim 12, wherein the precious metal is selected from the group consisting of rhodium, rhenium, ruthenium, platinum, palladium, osmium, iridium, gold and mixtures thereof.

14. The process of claim 12, wherein the acetic acid is derived from a carbonaceous material selected from the group consisting of oil, coal, natural gas and biomass.

15. The process of claim 12, wherein the rate of production of grams of ethanol per kilogram of catalyst per hour is at least 100 gₑₜₕₐₙₒₗ/kg_{catalyst}.

16. The process of claim 12, wherein the rate of production of grams of ethanol per kilogram of precious metal per hour is at least 1000 gₑₜₕₐₙₒₗ/kg_{precious metal}.

17. The process of claim 12, wherein acetic acid conversion is at least 80% and acetic acid selectivity to ethanol is at least 80%.

18. The process of claim 12, wherein hydrogenation is performed in a vapor phase at a temperature from 125 to 350° C., a pressure of 10 kPa to 3000 kPa, and a hydrogen to acetic acid molar ratio of at least 4:1.

19. A process for forming a catalyst for hydrogenating acetic acid and/or an ester thereof to form ethanol, the process comprising the steps of:
providing a pellet comprising a support and at least one metal selected from the group consisting of tungsten, molybdenum, vanadium, niobium, cobalt, tin, tantalum, and mixtures and oxides thereof, wherein the packing density of the pellet is less than 0.55 g/cm³, and
impregnating the pellet with a precious metal selected from the group consisting of rhodium, rhenium, ruthenium, platinum, palladium, osmium, iridium, gold and mixtures thereof.

20. The process of claim 19, wherein the packing density of the pellet is from 0.01 to 0.55 g/cm³.
